SEQUENCE LISTING (39 sequences)

SEQ ID NO: 1 - protein, 235 aa, synthetic construct
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGGGGSGGGGSGGGGSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIK

SEQ ID NO: 2 - protein, 10 aa, Homo sapiens
GGGSSGGGSG

SEQ ID NO: 3 - protein, 12 aa, synthetic construct
ESKYGPPCPPCP

SEQ ID NO: 4 - protein, 12 aa, Homo sapiens
ESKYGPPCPSCP

SEQ ID NO: 5 - protein, 22 aa, synthetic construct
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 22 aa, Homo sapiens
MLLLVTSLLLCELPHPAFLLIP

SEQ ID NO: 27 - protein, 24 aa, Homo sapiens
LEGGGEGRGSLLTCGDVEENPGPR

SEQ ID NO: 28 - protein, 354 aa, Homo sapiens
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 643 aa, synthetic construct
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGGGGSGGGGSGGGGSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 665 aa, synthetic construct
MLLLVTSLLLCELPHPAFLLIPEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGGGGSGGGGSGGGGSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 1046 aa, synthetic construct
MLLLVTSLLLCELPHPAFLLIPEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGGGGSGGGGSGGGGSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 32 - protein, 447 aa, synthetic construct
TDFTLTISSLEPEDFAVYYCLQWSSNPLTFGGGTKVEIKESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 114 aa, synthetic construct
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSS

SEQ ID NO: 34 - protein, 107 aa, synthetic construct
SEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIK

SEQ ID NO: 35 - protein, 18 aa, synthetic construct
GSTSGSKGPLGLAGATKG

SEQ ID NO: 36 - protein, 18 aa, synthetic construct
GSTSGSKGPKGLKGATKG

SEQ ID NO: 37 - protein, 14 aa, synthetic construct
GGGGSGGGGSGGGG

SEQ ID NO: 38 - protein, 234 aa, synthetic construct
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGSTSLGLAGATKGSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIK

SEQ ID NO: 39 - protein, 239 aa, synthetic construct
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGSTSGSKGPKGLKGATKGSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIK

(19) United States
(12) Patent Application Publication
(10) Pub. No.: US 2024/0148826 A1
(43) Pub. Date: May 9, 2024

(54) CHIMERIC ANTIGEN RECEPTORS AND METHODS FOR REDUCING TOXICITY

(71) Applicant: City of Hope, Duarte, CA (US)

(72) Inventors: Xiuli Wang, Temple City, CA (US); Stephen J. Forman, Duarte, CA (US); Lawrence Stern, Duarte, CA (US); Christine E. Brown, Duarte, CA (US); Joseph Cohen, Duarte, CA (US)

(21) Appl. No.: 18/329,365

(22) Filed: Jun. 5, 2023

Related U.S. Application Data
(63) Continuation of application No. 16/964,471, filed on Jul. 23, 2020, now Pat. No. 11,701,405.
(60) Provisional application No. 62/622,583, filed on Jan. 26, 2018.

(51) Int. Cl. A61K 38/17; A61K 35/17; C07K 16/18; C12N 15/86

(52) U.S. Cl. CPC A61K 38/177; A61K 35/17; C07K 16/18; C12N 15/86; C07K 2317/622

(57) ABSTRACT

Nucleic acid molecules that include a nucleotide sequence encoding a chimeric antigen receptor (CAR) and a nucleotide sequence encoding a protease sensitive scFv, wherein the chimeric antigen receptor comprises: an scFv targeting a tumor antigen, a spacer, a transmembrane domain, a co-stimulatory domain, and a CD3 ζ signaling domain; and the protease-sensitive scFv and the scFv target the same tumor antigen are described.

### CLAIM OF PRIORITY

This application is a continuation of U.S. application Ser. No. 16/964,471, filed on Jul. 23, 2020, which is the U.S. National Phase Application under 35 U.S.C. § 371 of International Application No. PCT/US2019/015252, filed on Jan. 25, 2019, which claims the benefit of U.S. Provisional Application Ser. No. 62/622,583, filed on Jan. 26, 2018. The entire contents of the foregoing are incorporated herein by reference.

### SEQUENCE LISTING

This application contains a Sequence Listing that has been submitted electronically as an XMIL file named 40056-0039002_SL_ST26.xml. The XMIL file, created on Jun. 5, 2023, is 39,847 bytes in size. The material in the XML file is hereby incorporated by reference in its entirety.

### BACKGROUND

Cancer initiating/stem cells (CSCs) (also known as tumor stem cells [TSCs]) are a distinct and highly motile and aggressive subpopulation of cells that reside within tumors, with the capacity to proliferate, differentiate and seed tumors1. Initially identified in acute myeloid leukemia (AML), CSCs have also been identified in solid tumors. TSCs are resistant to radio/chemotherapy and evidence suggests that they may be responsible for disease relapse. Complete elimination and prevention of tumor recurrence and growth relies on the eradication of CSCs, which remains challenging for the treatment of cancers, including hematopoietic malignancies. The CD44 transmembrane receptor is a cell adhesion glycoprotein that binds to components of the extracellular matrix (ECM) such as osteopontin, hyaluronan, collagen, laminin and fibronectin. CD44 has many biological functions including cellular growth, survival, and differentiation. Evidence also suggests that it is involved in tumor migration and metastasis. CD44 is widely expressed in a variety of cancers, and is a biomarker for CSCs. A splice variant of CD44, CD44 variant 6 (CD44v6), is overexpressed on malignant hematopoietic cells including AML, B cell malignancies and multiple myeloma (MM), especially in late stage diseases. CD44v6 plays an important role in proliferation, homing, and metastasis of tumor stem cells.

While CD44v6 appears to be an excellent target for CAR T cell therapy, its expression in human keratincytes and other tissues, as well as in monocytes, presents a challenge to CAR T cell or targeted antibody mediated therapy due to on-target off-tumor toxicity. Previous efforts using antibody drug conjugates employing bivatuzumab (a CD44v6 specific antibody) resulted in skin toxicity and fatalities from toxicity related events. To address on-target off-tumor toxicity, Casucci and colleagues offer the first preclinical studies using CAR-T cells that incorporate a suicide gene to target CD44v6 in AML and MM.

### SUMMARY

Described herein is a nucleic acid molecule comprising a nucleotide sequence encoding a chimeric antigen receptor (CAR) and a nucleotide sequence encoding a protease sensitive scFv, wherein the chimeric antigen receptor comprises: an scFv targeting a tumor antigen, a spacer, a transmembrane domain, a co-stimulatory domain, and a CD3 ζ signaling domain; and the protease-sensitive scFv and the scFv target the same tumor antigen. In some cases, the scFv within the CAR has VH and VL sequences that are identical to that of the protease sensitive CAR. The protease sensitive CAR further include a protease sensitive amino acid sequence (e.g., an MMP-2 or MMP-9 sensitive amino acid sequence) between the VH and VL sequences.

Described herein are methods for using CD44v6 targeted CAR T cells to treat a variety of cancers while reducing on-target, off-tumor toxicity. The methods entail the use of CD44v6 CAR T cells that secrete a soluble, protease-susceptible CD44v6 scFv that can block CAR binding in healthy tissue, but will be cleaved by cancer-specific proteases (e.g., MMP-2 or MMP-9) in the tumor site, allowing for CAR T cell binding and activation. Thus, described herein is a population of T cells expressing both a CD44v6-targeted CAR and a protease sensitive CD44v6 scFv.

In various embodiments: the protease sensitive scFv is sensitive to MMP-2 or MMP-9; the protease sensitive scFv comprises a V_{H} domain and a V_{L} domain joined by a protease-sensitive linker; the scFv and the protease-sensitive scFv have the same CDR sequences; the nucleic acid molecule comprises a nucleotide sequence encoding a T2A skip sequence; the nucleotide sequence encoding a T2A skip sequence is located between the nucleotide sequence encoding the chimeric antigen receptor and the nucleotide sequence encoding a protease sensitive; the tumor antigen is CD44v6; the scFv comprises the amino acid sequence of SEQ ID NO:1; and the V_{H} domain of the protease sensitive scFv comprises SEQ ID NO: 33 and the V_{L} domain of the protease sensitive scFv comprises SEQ ID NO: 34.

In various embodiments, the CAR comprises: a transmembrane domain selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; a costimulatory domain selected from: a 4-IBB costimulatory domain or a variant thereof having 1-5 amino acid modifications and an CD28 costimulatory domain or a variant thereof having 1-5 amino acid modifications; a CD3ζ signaling domain of a variant thereof having 1-5 amino acid modifications; and a spacer region having 20-150 amino acids located between the scFv and the transmembrane domain.

In various embodiments: The nucleic acid molecule of claim 1, wherein the costimulatory domain is selected from the group consisting of: a 4-IBB costimulatory domain and variants thereof having 1-5 amino acid modifications; the transmembrane domain is a CD4 transmembrane domain or variant thereof having 1-5 amino acid modifications; the transmembrane domain is a CD4 transmembrane domain; the CAR comprises two different costimulatory domains selected from the group consisting of: a CD28 costimulatory domain or a variant thereof having 1-5 amino acid modifications, a 4-IBB costimulatory domain or a variant thereof having 1-5 amino acid modifications and an OX40 costimulatory domain or a variant thereof having 1-5 amino acid modifications; the chimeric antigen receptor comprises a CD44v6-binding scFv having the amino acid sequence of SEQ ID NO:1 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3 ζ transmembrane domain or a variant thereof having 1-2 amino acid modifications; a 4-IBB costimulatory domain; or a variant thereof having 1-2 amino acid modifications; and CD3 ζ signaling domain of a variant thereof having 1-2 amino acid modifications; and a spacer region having 20-150 amino acids located between the scFv and the transmembrane domain; the spacer region comprises an amino acid sequence selected from the group consisting of SEQ ID NOs: 2-12 or a variant thereof having 1-5 amino acid modifications; the spacer comprises an IgG hinge region; the spacer comprises 10-50 amino acids; the 4-1BB costimulatory domain comprises the amino acid sequence of SEQ ID NO: 24 or a variant thereof having 1-5 amino acid modifications; the CD3ζ signaling domain comprises the amino acid sequence of SEQ ID NO:21; a linker of 3 to 15 amino acids is located between the costimulatory domain and the CD3 ζ signaling domain or variant thereof, and the CAR comprises the amino acid sequence of SEQ ID NO: 29 or 30 or a variant thereof having 1-5 amino acid modifications.

Also described here is an expression vector and a viral vector comprising the nucleic acid molecules described herein. Also described is a population of human T cells transduced by a vector comprising a nucleic acid molecule described herein. Also described is a method of treating cancer in a patient comprising administering a population of autologous or allogeneic human T cells transduced by a vector comprising a nucleic acid molecule described herein. In embodiments of the method: the population of human T cells comprise central memory T cells; MMP-2 or MMP-9 is present in the tumor microenvironment; the patient is suffering from a leukemia or lymphoma; and the patient is suffering from AML or multiple myeloma.

The approach used for CD44v6 CAR can be used with any target for treating cancer where the tumor microenvironment has proteases such as MMP-2 or MMP-9.

In various embodiments: the chimeric antigen receptor comprises: a CD44v6 scFv (e.g., an scFv comprising the amino acid sequence EVQLVESGGGLVKPGGSLRLSCAASGFTFSSYDMSWVRQAPGKGLEWVSTISSG GSYTYYLDSIKGRFTISRDNAKNSLYLQMNSLRAEDTAVYYCARQGLDYWGRG TLVTVSSGGGGSGGGGSGGGGSEIVLTQSPATLSLSPGERATLSCSASSSINYIYW YQQKPGQAPRLLIYLTSNLASGVPARFSGSGSGTDFTLTISSLEPEDFAVYYCLQW SSNPLTFGGGTKVEIK (SEQ ID NO:1) with up to 10 single amino acid substitutions); a spacer region; a CD28 transmembrane domain; a CD28 co-signaling domain; and a CD3 ζ signaling domain; the chimeric antigen receptor comprises: a CD44v6 scFv; a spacer region; a CD4 transmembrane domain; a 4-1BB co-signaling domain; and a CD3 ζ signaling domain; the chimeric antigen receptor comprises: a CD44v6 scFv; a spacer region comprising an amino acid sequence selected from SEQ ID Nos:2-5 and 9-12; a CD4 transmembrane domain; a 4-1BB co-signaling domain; and a CD3 ζ signaling domain; the chimeric antigen receptor comprises: a CD44v6 scFv; a spacer region comprising an amino acid sequence selected from SEQ ID Nos:2-5 and 9-12; a CD28 transmembrane domain; a CD28 co-signaling domain; and a CD3 ζ signaling domain; the chimeric antigen receptor comprises: a CD44v6 scFv; a spacer a spacer comprising an amino acid sequence selected from SEQ ID Nos:2-5 and 9-12; CD4 transmembrane domain; a 4-1BB co-signaling domain; and CD3 ζ signaling domain; the chimeric antigen receptor comprises: a spacer a spacer comprising an amino acid sequence selected from SEQ ID Nos:2-5 and 9-12; a CD28 transmembrane domain; a CD28 co-signaling domain; and a CD3 ζ signaling domain; the chimeric antigen receptor comprises an amino acid sequence at least 95% identical to an amino acid sequence selected from: SEQ ID NOs: 20-40; the chimeric antigen receptor comprises an amino acid sequence identical to an amino acid sequence selected from: SEQ ID NOs: 29-30; the chimeric antigen receptor comprises an amino acid sequence identical to an amino acid sequence selected from: SEQ ID NOs: 29-40, each with no more than 5 single amino acid substitutions; at least 20%, 30%, or 40% of the transduced human T cells are central memory T cells; at least 30% of the transduced human T cells are CD4+ and CD62L+ or CD8+ and CD62L+; the population of human T cells are autologous to the patient; and the population of human T cells are allogenic to the patient.

The protease sensitive scFv can comprise the sequence of SEQ ID NO:1 wherein the linker (GGGGSGGGGSGGGG; SEQ ID NO: 37) is replaced by a protease sensitive linker, for example, a linker sensitive to MMP-2 (e.g., GSTSGSKGPLGLAGATKG; SEQ ID NO: 35) or MMP-9 (e.g., GSTSGSKGPKGLKGATKG; SEQ ID NO: 36).

### DESCRIPTION OF DRAWINGS

FIG. 1: CD44v6R-41BBZ-T2A-huEGFRt construct contained in SIN lentiviral vector. Diagram of the CD44v6R:41BB:zeta-T2A-EGFRt construct, where the CD44v6-specific ScFv, IgG4 Fc hinge, CD4 transmembrane, 41BB and CD3z cytoplasmic signaling domains of the CD44v6R:41BB:zeta CAR, as well as the T2A ribosome skip and truncated huEGFR sequences are indicated. This vector, which expresses the CAR used in the studies described below, does not express a soluble scFv targeted to CD44v6.

FIG. 2 Purification and expansion of CD8⁺T_{CM} derived CD44v6CAR/EGFRt T cells. (A) CD8⁺ central memory T cells (CD62L⁺CD45RO⁺CD8⁺; CD8⁺T_{CM}) were isolated from a healthy donor and transduced with lentivirus encoding CD44v6CAR/EGFRt after CD3/CD28 beads activation. (A) Gene modified cells were immunomagnetically purified after labeling with biotinylated Erbitux followed by anti-biotin microbeads and expanded in rapid expansion medium (REM) containing OKT3 and feeder cells in the presence of IL-2 (50 U/ml) and IL-15 (1 ng/ml). (B) Fold expansion of purified CD44v6CAR T cells after each cycle of REM stimulation.

FIG. 3 Phenotype of ex vivo expanded CD44v6CAR/EGFRt CD8⁺ T cells Gene modified cells were immunomagnetically purified after labeling with biotinylated Erbitux followed by anti-biotin microbeads and expanded in rapid expansion medium (REM) containing OKT3 and feeder cells in the presence of IL-2 (50U/ml) and IL-15 (1 ng/ml). After 2 cycles of in vitro expansion, T cells were labeled with antibodies against EGFRt (Erbitux), CD62L, CD28 and CD27. % Positive cells (closed histogram) over isotype controls (open histogram) are presented.

FIG. 4: CD44v6 expression on leukemic cells. AML (THP-1 and KG1a) and B cell lymphoma (LCL) cells were labeled with PE-conjugated CD44v6 Ab (clone 2F10) (closed histogram) and isotype control Ab (open histogram) and analyzed by flow cytometry. % Positive cells over isotype controls are presented.

FIG. 5: Cytolytic activity of CD44v6CAR/EGFRt CD8⁺ T cells After 2 cycles of in vitro expansion, CD44v6CAR/EGFRt CD8⁺ T cells were incubated for 4 hrs with 51Cr-labled THP-1 or LCL cells, or OKT3-expressing LCL (LCLOKT3) cells as positive targets and CD44v6 negative KG1a cells as negative targets at 25:1 E:T ratios. Mean percent cytotoxicity of triplicate wells that was normalized over LCL OKT3 is depicted.

FIG. 6: Anti-lymphoma effects of adoptively transferred CD8⁺T_{CM} derived CD44v6CAR/EGFRt T cells. 2×10⁶ CD19⁺ffluc⁺ lymphoblastoid cell lines (LCL) cells were injected (i.v) into NSG mice on day −3. Subsequently, 5×10⁶ CD8⁺T_{CM} derived CD44v6CAR T cells were intravenously infused into the tumor bearing mice on day 0. Recipient mice received intraperitoneal injection of irradiated human IL15 secreting NSO cells to support human T cell persistence. Tumor signals were monitored by biophotonic imaging. (A) Representative images on day 14 are presented. (B) Mean±SEM of phonton/sec from multiple mice are depicted.

FIG. 7: Anti-AML effects of adoptively transferred CD8⁺T_{CM} derived CD44v6CAR/EGFRt T cells 1.5×10⁶ GFPffluc⁺ AML cells (THP-1) were injected (i.v) into NSG mice on day −3. 5×10⁶ CD8⁺T_{CM} derived CD44v6CAR T cells were intravenously infused into the tumor bearing mice on day 0. Mice received no T cells or CD8⁺T_{CM} derived irrelevant CAR (CD19CAR) T cells from the same donor were used as negative controls. All recipient mice received intraperitoneal injection of irradiated human IL15 secreting NSO cells to support human T cell persistence. (A) Tumor signals were monitored by biophotonic imaging. (B) Means±SEM of phonton/sec from multiple mice are depicted.

FIG. 8: CD8⁺T_{CM} derived CD44v6 CAR/EGFRt. T cells interfere with human leukemia initiation in immunodeficient mice 20×10⁶ CD44v6CAR T cells or irrelevant CAR T cells (CD19CAR) derived from CD8+T_{CM} of the same donor were adoptively transferred (i.v) into NSG mice. 2 weeks post T cell infusion, 1.5×10⁶ GFPffluc⁺ THP-1 cells expressing CD44v6 were inoculated (i.v) into the mice. (A) Tumor signals were monitored by biophotonic imaging 7 and 14 days post tumor infusion. (B) % Human T cells in peripheral blood are depicted

FIG. 9: Amino acid sequence of a CD44v6 CAR. The amino acid sequence presented is the amino acid sequence of a CD44v6 CAR, including the signal sequence, together with the sequence of the truncated EGFR sequence used for monitoring CAR expression and the T2A ribosomal skip sequence that allows the CAR to be co-expressed, but not fused to the truncated EGFR sequence (SEQ ID NO:35). The immature CAR includes: GMCSFR signal peptide, a CD44v6 scFv, an IgG4 derived sequence that acts as a spacer, a CD4 transmembrane domain, a 4-IBB co-stimulatory domain that includes a LL to GG sequence alteration, a three Gly sequence, a CD3 Zeta stimulatory domain. The transcript also encodes a T2A ribosomal sequence and a truncated EGFR sequence that are not part of the CAR protein sequence. The mature CAR is identical to the immature CAR, but lacks the GMCSF signal peptide. The various domains are indicated.

FIG. 10: Amino acid sequences of CD44v6 scFv. (A) CD44v6 scFv with MMP-2 sensitive linker (SEQ ID NO:38). (B) CD44v6 scFv with MMP-9 sensitive linker (SEQ ID NO: 39). (C) CD44v6 scFv V_{H} domain (SEQ ID NO:33). (D) CD44v6 scFv V_{L} domain (SEQ ID NO:34).

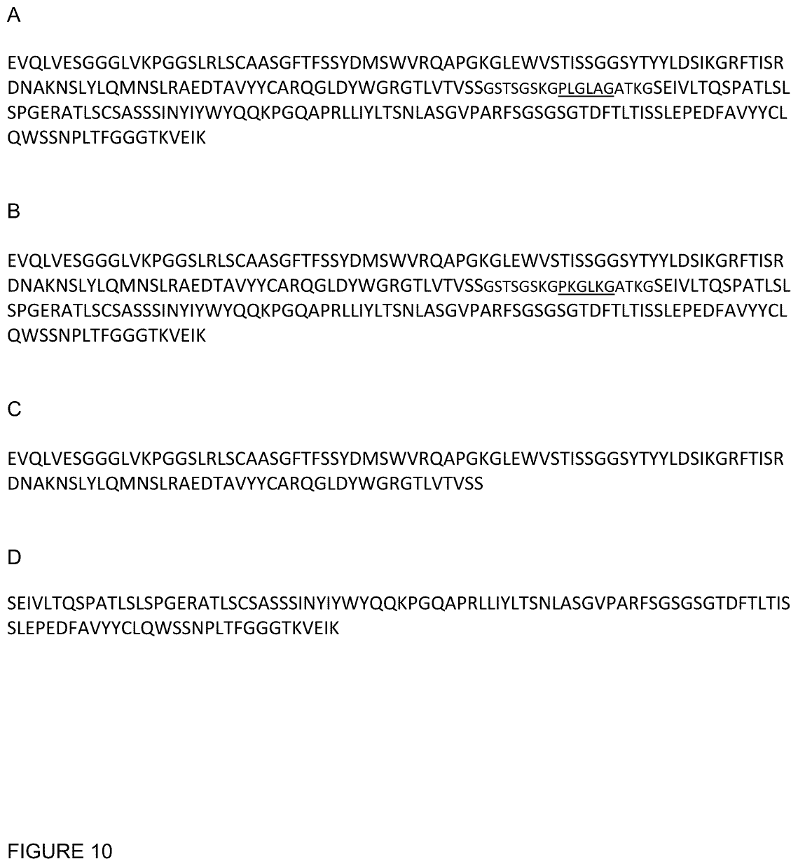

### DETAILED DESCRIPTION

### CD44v6 Targeted CAR

The CD44v6-targeted CAR described herein include a CD44v6-targeting scFv (e.g., an (e.g., an scFv comprising the amino acid sequence

(SEQ ID NO: 1)
EVQLVESGGGLVKPGGSLRLSCAASGFTFSSYDMSWVRQAPGKGLEWVST
ISSGGSYTYYLDSIKGRFTISRDNAKNSLYLQMNSLRAEDTAVYYCARQG
LDYWGRGTLVTVSSGGGGSGGGGSGGGGSEIVLTQSPATLSLSPGERATL
SCSASSSINYIYWYQQKPGQAPRLLIYLTSNLASGVPARFSGSGSGTDFT
LTISSLEPEDFAVYYCLQWSSNPLTFGGGTKVEIK
or comprising the heavy chain sequence
(SEQ ID NO: 33)
EVQLVESGGGLVKPGGSLRLSCAASGFTFSSYDMSWVRQAPGKGLEWVST
ISSGGSYTYYLDSIKGRFTISRDNAKNSLYLQMNSLRAEDTAVYYCARQG
LDYWGRGTLVTVSS
and the light chain sequence
(SEQ ID NO: 34)
EVQLVESGGGLVKPGGSLRLSCAASGFTFSSYDMSWVRQAPGKGLEWVST
ISSGGSYTYYLDSIKGRFTISRDNAKNSLYLQMNSLRAEDTAVYYCARQG
LDYWGRGTLVTVSS.

Useful CD44v6 CAR consist of or comprises the amino acid sequence of SEQ ID NO:29 (mature CAR lacking a signal sequence, T2A skip sequence and EGFRt) or the CD44v6 CAR consists of or comprises the amino acid sequence of SEQ ID NO: 30 (immature CAR having a GMCSFRa signal sequence, but lacking T2A skip sequence and EGFRt). The CAR and can be expressed in a form that includes a signal sequence, e.g., a human GM-CSF receptor alpha signal sequence (MLLLVTSLLLCELPHPAFLLIP; SEQ ID NO:26). The CAR can be expressed with additional sequences that are useful for monitoring expression, for example a T2A skip sequence and a truncated EGFRt. Thus, the CAR can comprise or consist of the amino acid sequence of any of SEQ ID Nos: 29-30 or can comprise or consist of an amino acid sequence that is at least 95%, 96%, 97%, 98% or 99% identical to any of SEQ ID Nos: 29-30. The CAR can comprise or consist of the amino acid sequence of any of SEQ ID Nos: 29-30 with up to 1, 2, 3, 4 or 5 amino acid changes (preferably conservative amino acid changes).

### Spacer Region

The CAR described herein can include a spacer located between the CD44v6 targeting domain (i.e., a gp120-targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + | 22 aa | ESKYGPPCPPCPGGGSSGGGSG (SEQ ID NO: 5) |
| linker | | |
| CD28 hinge | 39 aa | IEVMYPPPYLDNEKSNGTIIHVKGKHLCPSPLFPGPSKP |
| | | (SEQ ID NO: 6) |
| CD8 hinge-48aa | 48 aa | AKPTTTPAPRPPTPAPTIASQPLSLRPEACRPAAGGAVH |
| | | TRGLDFACD (SEQ ID NO: 7) |
| CD8 hinge-45aa | 45aa | TTTPAPRPPTPAPTIASQPLSLRPEACRPAAGGAVHTRG |
| | | LDFACD (SEQ ID NO: 8) |
| IgG4(HL-CH3) | 129 aa | ESKYGPPCPPCPGGGSSGGGSGGQPREPQVYTLPPSQEE |
| (includes 5228P | | MTKNQVSLTCLVKGFYPSDIAVEWESNGQPENNYKTTPP |
| in hinge) | | VLDSDGSFFLYSRLTVDKSRWQEGNVFSCSVMHEALHNH |
| | | YTQKSLSLSLGK (SEQ ID NO: 9) |
| IgG4(L235E, N297Q) | 229 aa | ESKYGPPCPSCPAPEFEGGPSVFLFPPKPKDTLMISRTP |
| | | EVTCVVVDVSQEDPEVQFNWYVDGVEVHQAKTKPREEQF |
| | | QSTYRVVSVLTVLHQDWLNGKEYKCKVSNKGLPSSIEKT |
| | | ISKAKGQPREPQVYTLPPSQEEMTKNQVSLTCLVKGFYP |
| | | SDIAVEWESNGQPENNYKTTPPVLDSDGSFFLYSRLTVD |
| | | KSRWQEGNVFSCSVMHEALHNHYTQKSLSLSLGK |
| | | (SEQ ID NO: 10) |
| IgG4(5228P, L235E, | 229 aa | ESKYGPPCPPCPAPEFEGGPSVFLFPPKPKDTLMISRTP |
| N297Q) | | EVTCVVVDVSQEDPEVQFNWYVDGVEVHQAKTKPREEQF |
| | | QSTYRVVSVLTVLHQDWLNGKEYKCKVSNKGLPSSIEKT |
| | | ISKAKGQPREPQVYTLPPSQEEMTKNQVSLTCLVKGFYP |
| | | SDIAVEWESNGQPENNYKTTPPVLDSDGSFFLYSRLTVD |
| | | KSRWQEGNVFSCSVMHEALHNHYTQKSLSLSLGK |
| | | (SEQ ID NO: 11) |
| IgG4(CH3) | 107 aa | GQPREPQVYTLPPSQEEMTKNQVSLTCLVKGFYPSDIAV |
| | | EWESNGQPENNYKTTPPVLDSDGSFFLYSRLTVDKSRWQ |
| | | EGNVFSCSVMHEALHNHYTQKSLSLSLGK |
| | | (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3).

The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTT PPVLDSDGSFFLYSRLTVDKSRWQEGNVFSCSVMHEALHNHYTQKSLSLSLGK (SEQ ID NO:12). Thus, the entire linker/spacer region can comprise the sequence: ESKYGPPCPPCPGGGSSGGGSGGQPREPQVYTLPPSQEEMTKNQVSLTCLVKGFY PSDIAVEWESNGQPENNYKTTPPVLDSDGSFFLYSRLTVDKSRWQEGNVFSCSV MHEALHNHYTQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27aa | FWVLVVVGGVLACYSLLVTVA |
| | | | FIIFWV (SEQ ID NO: 14) |
| CD28(M) | NM_006139 | 28aa | MFWVLVVVGGVLACYSLLVTV |
| | | | AFIIFWV (SEQ ID NO: 15) |
| CD4 | M35160 | 22aa | MALIVLGGVAGLLLFIGLGIFF |
| | | | (SEQ ID NO: 16) |
| CD8tm | NM_001768 | 21aa | IYIWAPLAGTCGVLLLSLVIT |
| | | | (SEQ ID NO: 17) |
| CD8tm2 | NM_001768 | 23aa | IYIWAPLAGTCGVLLLSLVITLY |
| | | | (SEQ ID NO: 18) |
| CD8tm3 | NM_001768 | 24aa | IYIWAPLAGTCGVLLLSLVITLYC |
| | | | (SEQ ID NO: 19) |
| 41BB | NM_001561 | 27aa | IISFFLALTSTALLFLLFFLTLRF |
| | | | SVV (SEQ ID NO: 20) |

### Costimulatory Domain

The costimulatory domain can be any domain that is suitable for use with a CD3ζ signaling domain. In some cases, the costimulatory domain is a CD28 costimulatory domain that includes a sequence that is at least 90%, at least 95%, at least 98% identical to or identical to: RSKRSRGGHSDYMNMTPRRPGPTRKHYQPYAPPRDFAAYRS (SEQ ID NO:23; LL to GG amino acid change double underlined). In some cases, the CD28 co-signaling domain has 1, 2, 3, 4 of 5 amino acid changes (preferably conservative and preferably not in the underlined GG sequence) compared to SEQ ID NO:23. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 or 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples of Costimulatory Domains

| Name | Accession | Length | Sequence |
|---|---|---|---|
| CD3ζ | J04132.1 | 113 aa | RVKFSRSADAPAYQQGQNQLYNE |
| | | | LNLGRREEYDVLDKRRGRDPEMG |
| | | | GKPRRKNPQEGLVNELQKDKMAE |
| | | | AYSEIGMKGERRRGKGHDGLYQG |
| | | | LSTATKDTYDALHMQALPPR |
| | | | (SEQ ID NO: 21) |
| CD28 | NM_006139 | 42aa | RSKRSRLLHSDYMNMTPRRPGPT |
| | | | RKHYQPYAPPRDFAAYRS |
| | | | (SEQ ID NO: 22) |
| CD28gg* | NM_006139 | 42aa | RSKRSRGGHSDYMNMTPRRPGPT |
| | | | RKHYQPYAPPRDFAAYRS |
| | | | (SEQ ID NO: 23) |
| 41BB | NM_001561 | 42 aa | KRGRKKLLYIFKQPFMRPVQTTQ |
| | | | EEDGCSCRFPEEEEGGCEL |
| | | | (SEQ ID NO: 24) |
| OX40 | | 42 aa | ALYLLRRDQRLPPDAHKPPGGGS |
| | | | FRTPIQEEQADAHSTLAKI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and in some cases a short linker consisting of 2-10, e.g., 3 amino acids (e.g., GGG) is positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRK NPQEGLYNELQKDKMAEAYSEIGMKGERRRGKGHDGLYQGLSTATKDTYDAL HMQALPPR (SEQ ID NO:21). In some cases, the CD3ζ signaling has 1, 2, 3, 4 of 5 amino acid changes (preferably conservative) compared to SEQ ID NO:21.

### Truncated EGFR

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHIL PVAFRGDSFTHTPPLDPQELDILKTVKEITGFLLIQAWPENRTDLHAFENLEIIRGR TKQHGQFSLAVVSLNITSLGLRSLKEISDGDVIISGNKNLCYANTINWKKLFGTSG QKTKIISNRGENSCKATGQVCHALCSPEGCWGPEPRDCVSCRNVSRGRECVDKC NLLEGEPREFVENSECIQCHPECLPQAMNITCTGRGPDNCIQCAHYIDGPHCVKT CPAGVMGENNTLVWKYADAGHVCHLCHPNCTYGCTGPGLEGCPTNGPKIPSIA TGMVGALLLLLVVALGIGLFM (SEQ ID NO:28). In some cases, the truncated EGFR has 1, 2, 3, 4 of 5 amino acid changes (preferably conservative) compared to SEQ ID NO:28.

### CD44v6 Targeted CAR

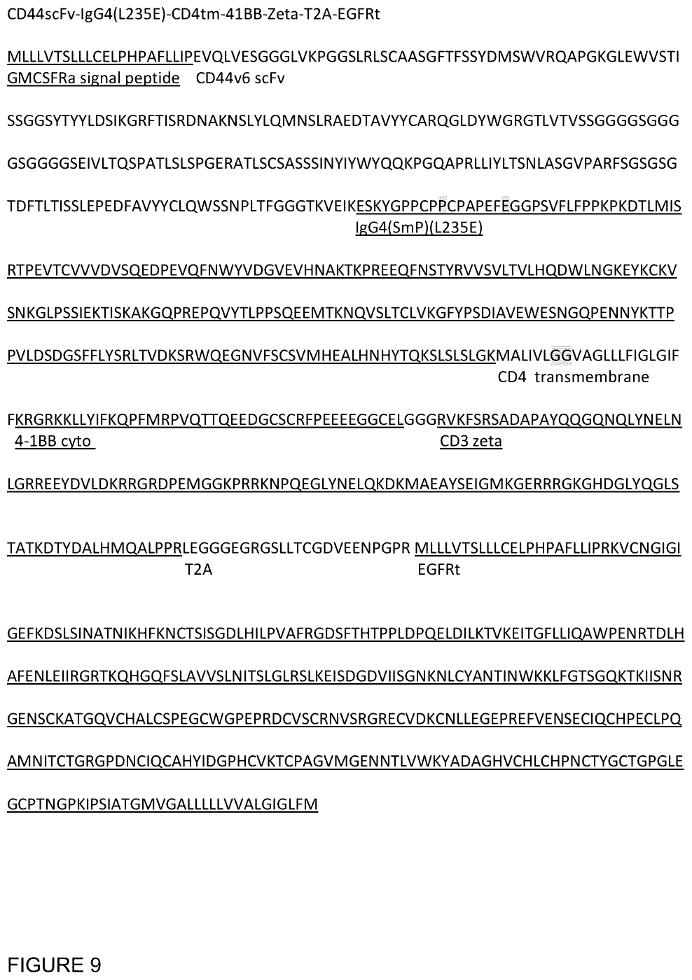

FIG. 9 depicts the amino acid sequence of a CD44v6 targeted CAR together with a truncated EGFR that can be used as a marker, which is joined to the CAR sequence via a T2A skip sequence. The truncated EGFR is expressed by cells that express the CAR and in this way serves as a marker and as a means to target CAR expressing cells. The sequence also includes a GMSCFRa signal sequence, that like the EGFRt sequence that are not present in the mature CAR as expressed on T cells. described herein. Useful CAR include hose that comprise or consist of the amino acid sequence of any of SEQ ID Nos: 29-30 or an amino acid sequence that is at least 95%, 96%, 97%, 98% or 99% identical to any of SEQ ID Nos: 29-30 or the amino acid sequence of any of SEQ ID Nos: 29-30 with up to 1, 2, 3, 4 or 5 amino acid changes (preferably conservative amino acid changes). In various embodiments: the population of human T cells are CD8+ cells.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

The CAR can include a sequence that is at least 90%, at least 95%, at least 98% identical to or identical to the mature amino acid sequence depicted in FIG. 9 (SEQ ID Nos: 29-40), either including or excluding the GMCSFRa signal sequence and either including or excluding the T2A ribosomal skip sequence and the truncated EGFRt).

In some cases, the CD44v6 CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the CD44v6CAR lentiviral vector can act as suicide gene to ablate the CAR+ T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an CD44v6 CAR as well as a non-immunogenic surface marker for in vivo detection, ablation, and potential ex vivo selection. The activated/genetically modified CD44v6 central memory T cells can be expanded in vitro with IL-2/IL-15 and then cryopreserved.

### Example 1: Preparation and Characterization of CD44v6 CAR

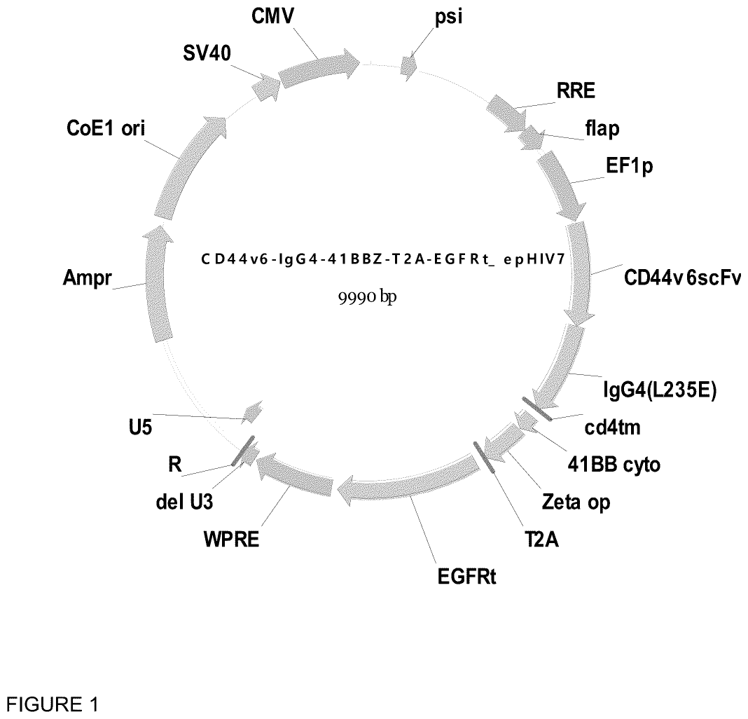

CD44v6R-41BBZ-T2A-huEGFRt construct contained in SIN lentiviral vector. A lentiviral construct was prepared using the approach described in Wang et al. (Blood 118:1255, 2011). FIG. 1 is a diagram of the CD44v6R:41BB:zeta-T2A-EGFRt construct, where the CD44v6-specific ScFv, IgG4 Fc hinge, CD4 transmembrane, 41BB and CD3z cytoplasmic signaling domains of the CD44v6R:41BB:zeta CAR, as well as the T2A ribosome skip and truncated huEGFR sequences are indicated. This vector, which expresses the CAR used in the studies described below, does not express a soluble scFv targeted to CD44v6.

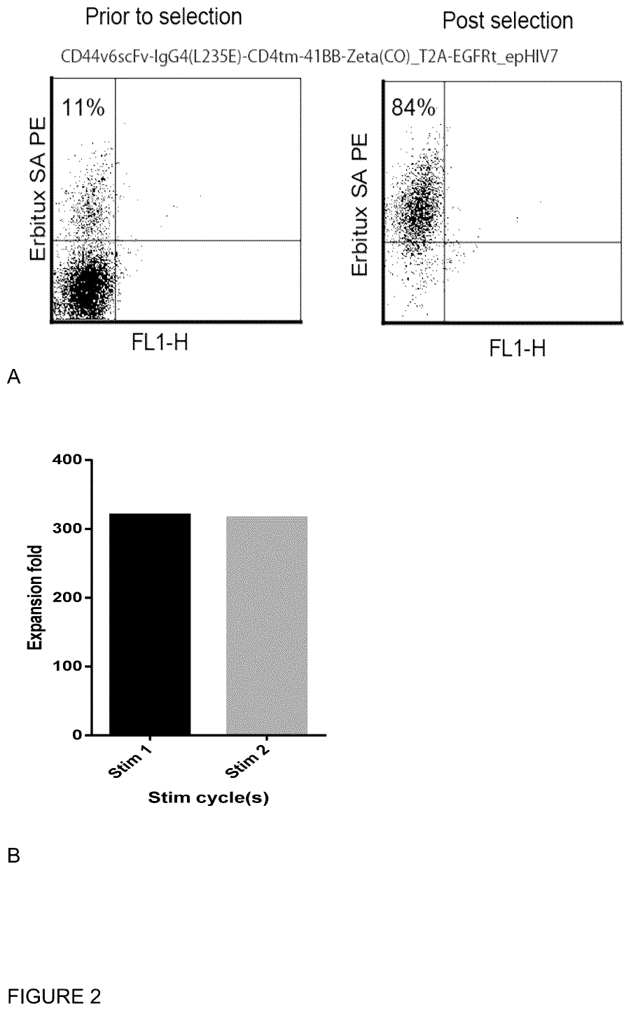

Purification and expansion of CD8⁺T_{CM} derived CD44v6CAR/EGFRt T cells. CD8⁺ central memory T cells (CD62L⁺CD45RO⁺CD8⁺; CD8⁺T_{CM}) were isolated from a healthy donor and transduced with lentivirus encoding CD44v6CAR/EGFRt after CD3/CD28 beads activation using the approach described in Wang et al. (Blood 117:1888, 2011). Gene modified cells were immunomagnetically purified after labeling with biotinylated Erbitux followed by anti-biotin microbeads and expanded in rapid expansion medium (REM) containing OKT3 and feeder cells in the presence of IL-2 (50 U/ml) and IL-15 (1 ng/ml) (FIG. 2A). FIG. 2B shows the fold expansion of purified CD44v6CAR T cells after each cycle of REM stimulation.

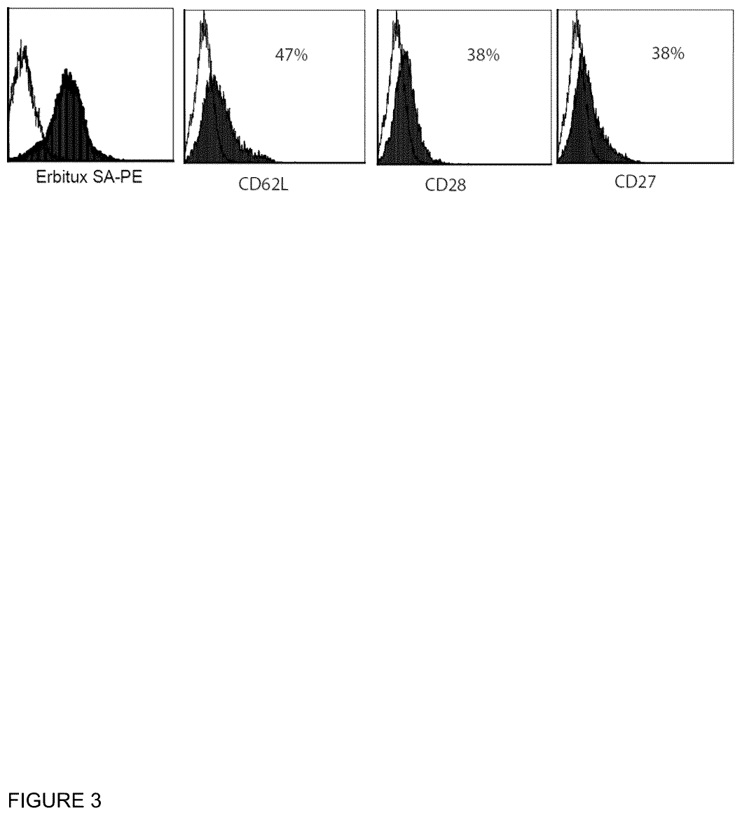

Phenotype of ex vivo expanded CD44v6CAR/EGFRt CD8⁺ T cells. Gene modified cells were immunomagnetically purified after labeling with biotinylated Erbitux followed by anti-biotin microbeads and expanded in rapid expansion medium (REM) containing OKT3 and feeder cells in the presence of IL-2 (50 U/ml) and IL-15 (1 ng/ml). After 2 cycles of in vitro expansion, T cells were labeled with antibodies against EGFRt (Erbitux), CD62L, CD28 and CD27. The results of this analysis are presented in FIG. 3 where the % Positive cells (closed histogram) over isotype controls (open histogram) are reported.

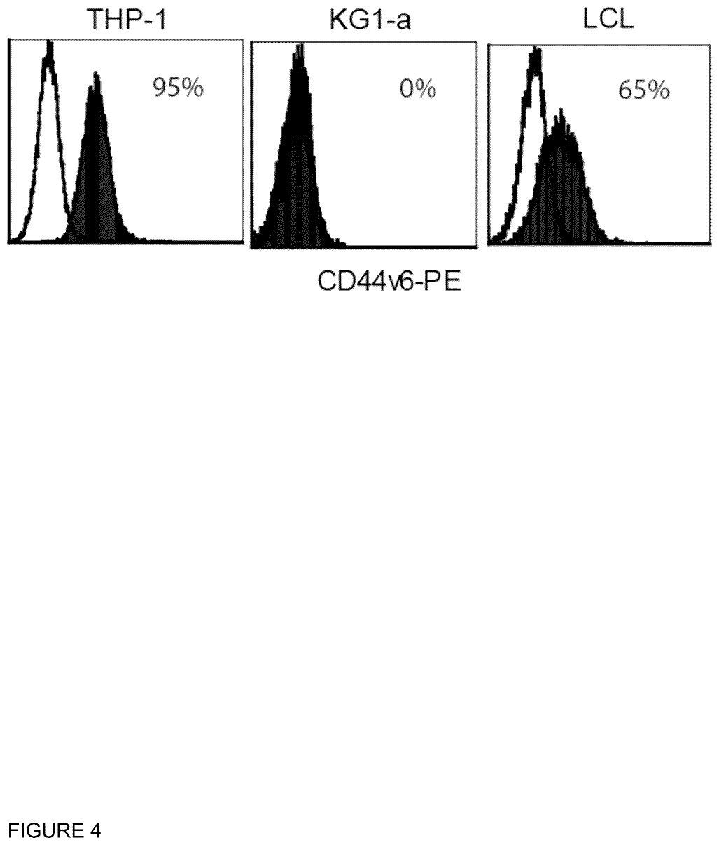

CD44v6 expression on leukemic cells. AML (THP-1 and KG1a) and B cell lymphoma (LCL) cells were labeled with PE-conjugated CD44v6 Ab (clone 2F10) and isotype control Ab and analyzed by flow cytometry. The results of this analysis are presented in FIG. 4 where the % Positive cells over isotype controls are presented.

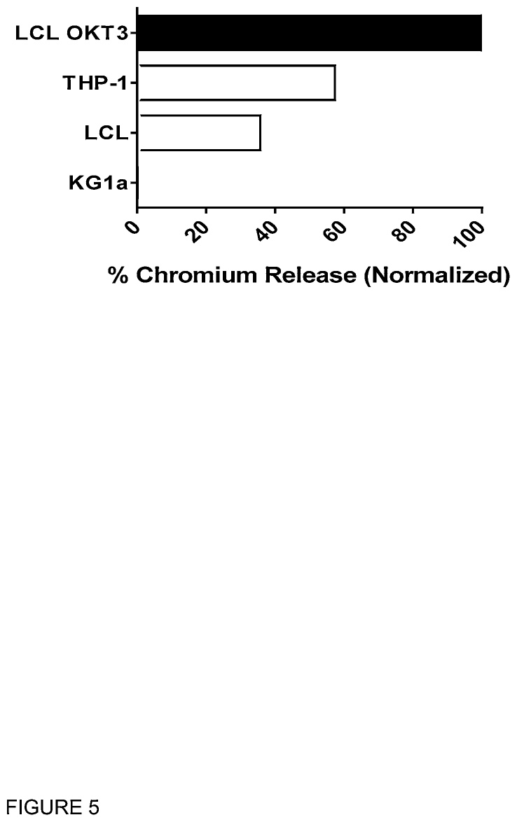

Cytolytic activity of CD44v6CAR/EGFRt CD8⁺ T cells. After 2 cycles of in vitro expansion, CD44v6CAR/EGFRt CD8⁺ cells were incubated for 4 hrs with 51Cr-labled THP-1 or LCL cells, or OKT3-expressing LCL (LCLOKT3) cells as positive targets and CD44v6 negative KG1a cells as negative targets at 25:1 E:T ratios. The results of this analysis are presented in FIG. 5.

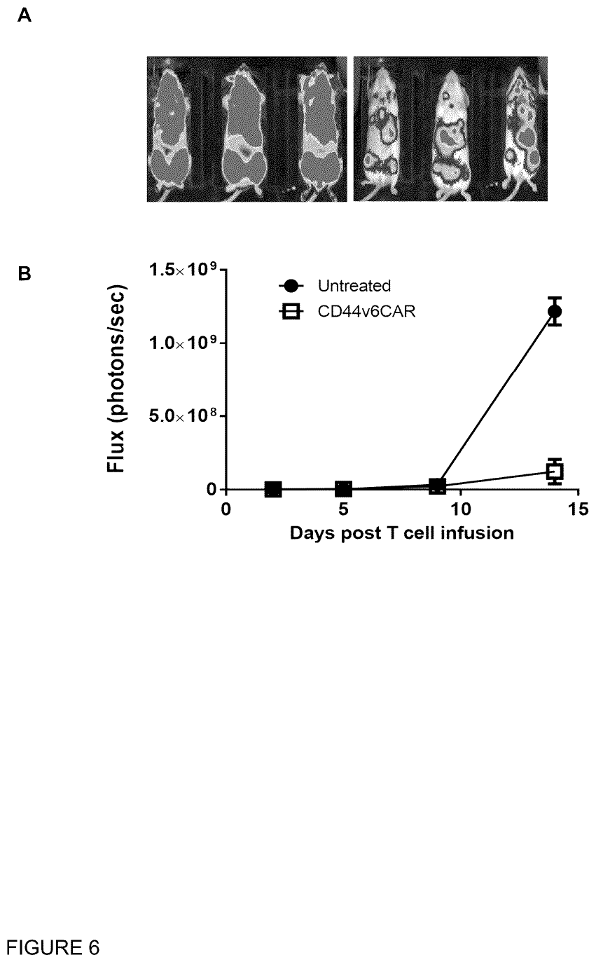

Anti-lymphoma effects of adoptively transferred CD8⁺T_{CM} derived CD44v6CAR/EGFRt T cells. The examine the anti-lymphoma effect of adoptively transferred CD8+TCM derived CD44v6CAR/EGFRt T cells, 2×10⁶ CD19⁺ffluc⁺ lymphoblastoid cell lines (LCL) cells were injected (i.v) into NSG mice on day −3. Subsequently, 5×10⁶ CD8⁺T_{CM} derived CD44v6CAR T cells were intravenously infused into the tumor bearing mice on day 0. Recipient mice received intraperitoneal injection of irradiated human IL15 secreting NSO cells to support human T cell persistence. Tumor signals were monitored by biophotonic imaging. The results of this analysis are presented in FIG. 6.

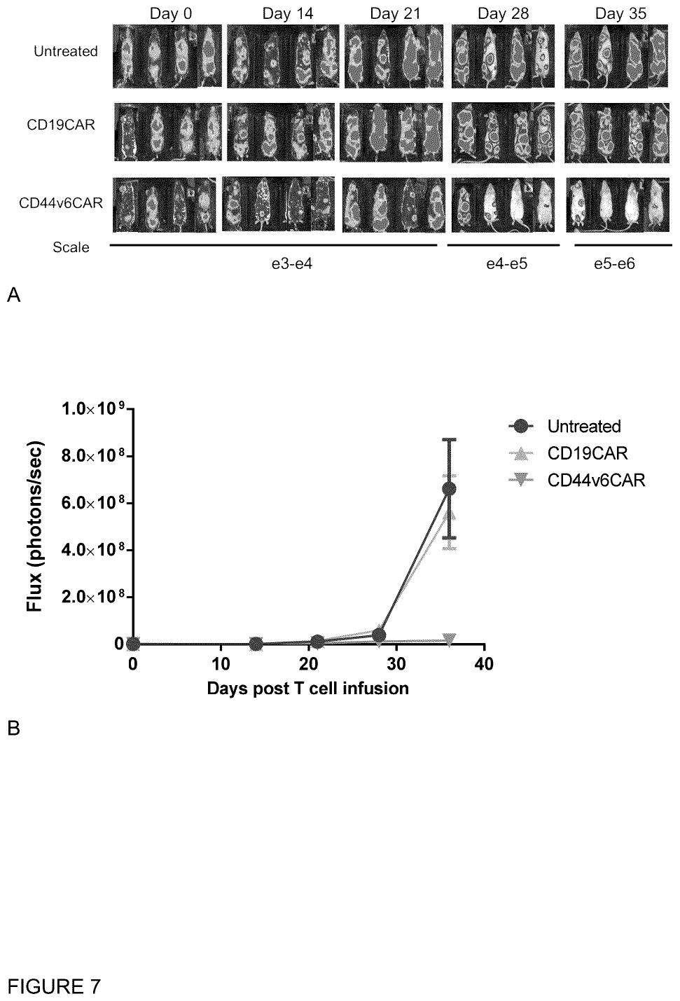

Anti-AML effects of adoptively transferred CD8⁺T_{CM} derived CD44v6CAR/EGFRt T cells. For this study, 1.5×10⁶ GFPffluc⁺ AML cells (THP-1) were injected (i.v) into NSG mice on day −3.5×10⁶ CD8⁺T_{CM} derived CD44v6CAR T cells were intravenously infused into the tumor bearing mice on day 0. Mice received no T cells or CD8⁺T_{CM} derived irrelevant CAR (CD19CAR) T cells from the same donor were used as negative controls. All recipient mice received intraperitoneal injection of irradiated human IL15 secreting NSO cells to support human T cell persistence. The results are presented in FIG. 7.

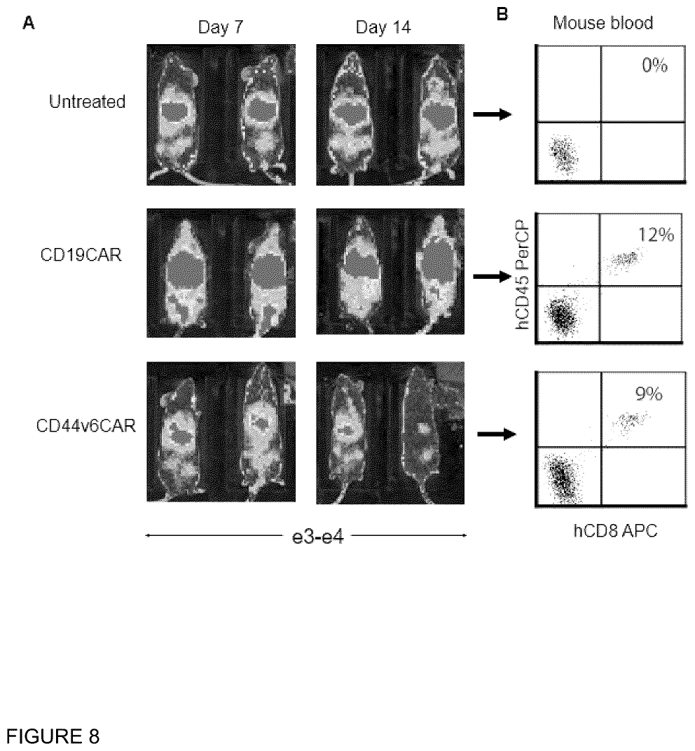

CD8⁺T_{CM} derived CD44v6 CAR/EGFRt T cells interfere with human leukemia initiation in immunodeficient mice. In this study, 20×10⁶ CD44v6CAR T cells or irrelevant CAR T cells (CD19CAR) derived from CD8+T_{CM} of the same donor were adoptively transferred (i.v) into NSG mice. 2 weeks post T cell infusion, 1.5×10⁶ GFPffluc⁺ THP-1 cells expressing CD44v6 were inoculated (i.v) into the mice. The results of this analysis are presented in FIG. 8.

Summary. The results of these studies suggest that targeting CD44v6 with CD44v6 CAR CD8+ T_{CM} cells can lead to potent anti-tumor activity upon adoptive transfer in a murine model and that CD44v6 CAR T cells are capable of interfering leukemia initiation by inhibiting leukemic stem cell homing and proliferation.

### Example 2: Generation of Protease Sensitive scFv

A selected scFv (e.g., the scFv used in the selected CAR) can be converted to a protease sensitive scFv by replacing the linker between the variable heavy chain and variable light chain portions with a linker that is sensitive to the selected protease, for example, MMP-2 or MMP-9, both of which are overexpressed and accumulated in various tumors. Yeast surface display, a genotype-phenotype linkage strategy for facile screening of protein libraries can be used to select scFv linkers that are appropriately susceptible to cleavage by MMP-2 and/or MMP-9 and allow for proper heavy and light chain association (Gai et al. 2007 Curr Opin Struct Biol 17:467-473). Because MMP-2 and MMP-9 are known to be promiscuous (Prudova et al. 2010 Mol Cell Proteomics 9:894-911), a library of candidate cleavage sites (residues G-P-X-X-X-X-X-A, 3.3×10⁷ variants) can be screened as the central portion of the scFv linker. Sequential fluorescence-activated cell sorting (FACS) experiments are employed to: (1) select linker candidates that yield scFvs with equivalent binding activity to the parent scFv, (2) select linker candidates that are cleaved by physiological concentrations of recombinant active MMP-2 and/or MMP-9, and (3) select linker candidates with equivalent serum stability to the parental linker. Successful linker candidates are screened for immunogenicity using the EpiSweep software package (Choi et al. 2017 Methods in Molecular Biology 1529: 375, 2017), with mutations made as needed to eliminate T cell epitopes. Once appropriate lead linker candidates are selected, the scFv can undergo affinity maturation using error-prone PCR (Zaccolo et al. 1996 Mol Biol 255:589-60324) followed by FACS to select for scFvs with improved binding affinity, an approach that has been employed successfully for scFvs in the past (Tillotson et al. 2013 Protein Eng Des Sel 26:101-112). Lead scFvs are cloned into a bicistronic construct encoding for the EF1α promoter, the soluble protease-susceptible scFv, a T2A ribosome skip sequence, and the parental CAR. Off-tumor toxicities of the CAR are evaluated in vitro and in appropriate murine models.

1. A nucleic acid molecule comprising:
a nucleotide sequence encoding a chimeric antigen receptor (CAR) comprising:
an scFv comprising a VH domain comprising SEQ ID NO: 33 and a VL domain SEQ ID NO:34, a spacer comprising an amino acid sequence of any one of SEQ ID NOs: 2-12, a transmembrane domain comprising an amino acid sequence of any one of SEQ ID NOs: 13-20, a co-stimulatory domain comprising an amino acid sequence of any one of SEQ ID NOs: 22-25, and a CD3 ζ signaling domain.

2. A nucleic acid molecule encoding a protease sensitive scFv comprising a VH domain comprising SEQ ID NO: 33 and a VL domain SEQ ID NO:34 joined by a protease-sensitive linker that is sensitive to MMP-2 or MMP-9.
